(19) United States
(12) Patent Application Publication
(10) Pub. No.: US 2010/0098655 A1
(43) Pub. Date: Apr. 22, 2010

(54) Aqueous Oligo- And Polyester Formulations

(75) Inventors: Frank-Peter Lang, Hattersheim (DE); Roman Morschhaeuser, Mainz (DE)

Correspondence Address: CLARIANT CORPORATION;INTELLECTUAL PROPERTY DEPARTMENT, 4000 MONROE ROAD, CHARLOTTE, NC, 28205, US

(73) Assignee: CLARIANT FINANCE (BVI) LIMITED, Tortola (VG)

(21) Appl. No.: 12/525,384

(22) PCT Filed: Jan. 29, 2008
(86) PCT No.: PCT/EP08/00647

§ 371 Date: Jul. 31, 2009

(30) Foreign Application Priority Data
Feb. 3, 2007 (DE) 10 2007 005 532.5

(51) Int. Cl. A61K 8/85; C08K 5/53; C11D 3/37; D06M 13/224

(52) U.S. Cl. 424/78.37; 524/130; 510/405; 252/8.81

(57) ABSTRACT

Aqueous formulations of soil release polyesters are claimed, which comprise from 50 to 90% by weight of soil release polyester and from 0.1 to 40% by weight of a phosphonic acid or of a phosphonate. The addition of the phosphonic acid or of the phosphonate allows the preparation of formulations of the soil release polyesters which are stable and have a low viscosity.

The invention relates to aqueous formulations of oligo- and polyesters and their use in detergents and cleaners, in the textile industry and in cosmetics.

Water-soluble or water-dispersible polyesters have been known for a long time. They are used in textile finishing for hydrophilization, for improving the moisture transport, for improving the ability of hydrophobic soilings (fats and oil) to be washed out and for improving the antistatic properties of polyester fabrics. Also known is their use as so-called soil release polymers in detergents and cleaners for textiles. Here, they serve to improve soil release from synthetic fibers, in particular from polyester fabrics and polyester blends. Furthermore, the use of certain water-soluble oligo- and polyesters in cosmetic preparations such as skin creams or shower gels is known. Here, they serve, for example, to improve the skin feel (skin conditioner).

These water-soluble or water-dispersible polyesters are polycondensates based on dicarboxylic acids and starting materials which have two or more hydroxyl groups. The dicarboxylic acid used is usually terephthalic acid. Besides these, further dibasic carboxylic acids, such as, for example, isophthalic acid, may be present. Furthermore, tricarboxylic acids can also be used (as crosslinkers). The starting materials with a plurality of hydroxyl groups (polyols) used are, for example, ethylene glycol, propylene glycol, butylene glycol, their dimers, trimers, oligomers or polymers. Components which have three or more hydroxyl groups, such as, for example, glycerol or pentaerythritol, may also be present. Monofunctional starting materials, such as, for example, methylpolyethylene glycols, are used as endcaps for controlling the molecular weight of the polyesters.

For the use of the polyesters as soil release polymers in liquid detergents and cleaners, these are supplied in undiluted form, i.e. as pasty or wax-like products, or in the form of aqueous preparations.

The undiluted products have the disadvantage that they first have to be melted so that they can then be metered into a liquid detergent or cleaner formulation in liquid, pourable or pumpable form.

However, heatable storage containers and pipelines have to be provided for this purpose, which represents not inconsiderable technical expenditure.

The standard commercially available aqueous preparations in turn have the disadvantage that they can only be produced with relatively low active ingredient contents. A further disadvantage of all aqueous polyester preparations is that they only have a very limited physical storage stability. Clear preparations as are absolutely necessary for producing clear detergents and cleaners become cloudy at elevated temperatures as prevail in the summer or in southern regions, even after a short time, and a voluminous, unattractive precipitate settles out. They can therefore become unusable even before or during transportation to the customer. Separating off the precipitate by means of filtration also only constitutes a temporary solution to the problem since such a precipitate can form again afterwards. The formation of a precipitate must thus here be avoided under all circumstances. By contrast, aqueous dispersions have a tendency toward phase separation, during which the polymer particles dispersed in water slowly sediment.

The PCT application 2005/006 344 describes the use of polycarboxylic acids for stabilizing oligo- and polyester formulations.

It is now an object of the present invention to provide aqueous preparations of oligo- and polyesters which have even better physical stability, thus do not become cloudy upon prolonged storage and at relatively high temperatures and are of low viscosity and which are therefore easy to store and to process by the user.

Surprisingly, it has now been found that this aim can be achieved through the addition of a phosphonic acid or salts thereof.

The invention provides aqueous formulations of oligo- and polyesters which comprise essentially 25 to 90% by weight of a soil release polyester and 0.1 to 40% by weight of a phosphonic acid or salts thereof.

The oligo- and polyesters for the preparation of the aqueous formulations may be water-soluble or water-dispersible and also nonionic or anionic. The molar mass of these polyesters is preferably less than or equal to 20 000, preferably less than or equal to 10 000 and particularly preferably less than or equal to 5000.

Nonionic oligo- and polyesters are described, for example, in the following patent specifications: U.S. Pat. No. 3,712,873, U.S. Pat. No. 3,959,230, U.S. Pat. No. 4,116,885, EP 0 442 101, DE 44 03 866, EP 253 567, EP 357 280 and DE 195 22 431. They can be composed on the basis of the following monomers:

Dihydric alcohols (glycols), in particular ethylene glycol; 1,2-propylene glycol; 1,3-propylene glycol; 1,2-butylene glycol; 2,3-butylene glycol; 1,4-butylene glycol; pentanediol; hexanediol, 3-methoxy-1,2-propylene glycol.

Polyhydric alcohols, in particular glycerol, pentaerythritol, oligoglycerols and alkoxylated secondary products thereof.

Addition products of preferably 1 to 5 mol of ethylene oxide and/or propylene oxide onto 1 mol of the aforementioned at least dihydric alcohols, such as, for example, ethylene diglycol, propylene diglycol, addition products of preferably 1 to 3 mol of ethylene oxide and/or propylene oxide onto 1 mol of glycerol, addition products of preferably 1 to 4 mol of ethylene oxide and/or propylene oxide onto pentaerythritol.

Polyalkylene glycols. These are preferably derived from ethylene oxide, propylene oxide, n-butylene oxide or isobutylene oxide. In this connection, these may be homopolymers, copolymers or terpolymers of said alkylene oxides. The copolymers may be block copolymers, random copolymers or alternating copolymers. Preference is given to using polyethylene glycol, polypropylene glycol or block copolymers thereof. These polyalkylene glycols preferably have molar masses of up to 4000 g/mol.

Alkyl polyalkylene glycols, in particular water-soluble addition products of preferably 5 to 80 mol of alkylene oxide(s) onto 1 mol of C₈-C₂₄-alcohols, C₆-C₁₈-alkylphenols or C₈-C₂₄-alkylamines. Preferred alkylene oxides are ethylene oxide, propylene oxide, butylene oxide, and mixtures thereof.

Aromatic dicarboxylic acids, in particular terephthalic acid, isophthalic acid. Preferably, the polyesters comprise up to 60% by mass, preferably up to 50% by mass, of terephthalic acid.

C₁-C₄-Alkyl polyalkylene glycols, where the parent polyalkylene glycols here preferably have molar masses of up to 4000 g/mol. Of suitability here is preferably methyl polyethylene glycol which contains up to 90, preferably up to 50 and particularly preferably up to 20, units of ethylene oxide.

Aliphatic dicarboxylic acids. Suitable aliphatic dicarboxylic acids contain e.g. 2 to 10 carbon atoms. Examples thereof are oxalic acid, malonic acid, succinic acid, glutaric acid, adipic acid, maleic acid, fumaric acid, itaconic acid and citraconic acid. They can be used individually or in a mixture.

Monohydroxymonocarboxylic acids, in particular glycolic acid, lactic acid, ω-hydroxystearic acid and ω-hydroxycaproic acid.

Monocarboxylic acids, such as, for example, benzoic acid, can be used as monofunctional starting materials for controlling the molecular weight.

Esters and anhydrides. The aforementioned carboxylic acids can also be used in the form of their esters or—if accessible—their anhydrides. Examples thereof are dimethyl terephthalate, diethyl terephthalate, diethyl oxalate, dimethyl adipate, phthalic anhydride, maleic anhydride, succinic anhydride.

Anionic oligo- and polyesters are described, for example, in U.S. Pat. No. 4,427,557; U.S. Pat. No. 4,721,580; U.S. Pat. No. 5,691,298; U.S. Pat. No. 5,700,386; U.S. Pat. No. 5,843,878; WO 96/18715; WO 95/02028; WO 95/02029 and EP 707627.

To prepare anionic oligo- and polyesters, besides the aforementioned components used for the preparation of nonionic polyesters, additionally e.g. hydroxyethanesulfonic acid, hydroxypropanesulfonic acid, reaction products thereof with alkylene oxides, preferably with ethylene oxide and/or propylene oxide, glycerin sulfoethyl ether, glycerin sulfopropyl ether, sulfoisophthalic acid and sulfobenzoic acid are condensed in.

Oligo- and polyesters which can be used according to the invention preferably have the following structure:

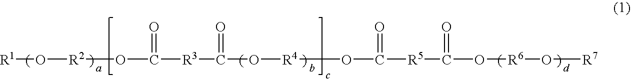

in which
R¹ and R⁷
are a linear or branched C₁-C₁₈-alkyl
R², R⁴, R⁶ independently of one another are alkylene, preferably ethylene, propylene, butylene and/or cycloalkylene, e.g. 1,4-cyclohexylene or 1,4-dimethylenecyclohexylene and mixtures thereof.
R³ and R⁵ are arylene or alkarylene, such as, for example, 1,4-phenylene, 1,3-phenylene, 1,2-phenylene, 1,8-naphthylene, 1,4-naphthylene, 2,2′-biphenylene, 4,4′-biphenylene; alkylene or alkenylene, such as, for example, methylene, ethylene, propylene, butylene, pentylene, hexylene; cycloalkylene, such as, for example, cyclohexylene
a, b and d are a number between 1 and 400
c is a number between 1 and 20.

Preference is given to oligo- and polyesters of formula (1) in which
R¹ and R⁷ are methyl and/or ethyl,
R², R⁴, R⁶ are ethylene, 1,2-propylene, or mixtures thereof
R³ and R⁵ are 1,4-phenylene and 1,3-phenylene and
a, b and d are a number between 1 and 100
c is a number between 1 and 10.

Preferred oligo- and polyesters are also those of formula (2)

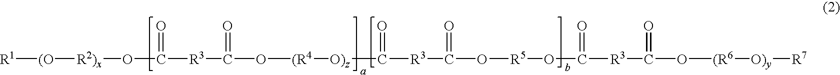

in which
R¹ and R⁷ are a linear or branched C₁-C₁₈-alkyl, R² and R⁶ are ethylene, R³ is 1,4-phenylene, 1,3-phenylene, R⁴ is ethylene, R⁵ is ethylene, 1,2-propylene or random mixtures of any desired composition of the two, x and y, independently of one another, are a number between 1 and 500, z is a number between 10 and 140, a is a number between 1 and 12, b is a number between 7 and 40.

Preferably, independently of one another, in formula (2),

R¹ and R⁷ are a linear or branched C₁-C₄-alkyl, x and y are a number between 3 and 45, z is a number between 18 and 70, a is a number between 2 and 5, b is a number between 8 and 12, a+b is a number between 12 and 35.

Besides the oligo- and polyesters, the aqueous formulations according to the invention comprise phosphonic acids or salts thereof for stabilization.

Suitable phosphonic acids or salts thereof are preferably organic phosphonic acids or salts thereof, preferably those with a molar mass of up to 20 000 g/mol.

Suitable phosphonic acids are, for example, the following compounds: (poly)phosphonic acid, aminotrimethylenephosphonic acid (ATMP), ethylenediaminetetramethylenephosphonic acid, hexamethylenediaminetetramethylenephosphonic acid, diethylenetriaminepentamethylenephosphonic acid, polyaminomethylenephosphonic acids, hexamethylenetriaminepenta-methylenephosphonic acid and hydroxyethylidene-1,1-diphosphonic acid and (poly)vinylphosphonic acid. A preferred phosphonic acid is hydroxyethylidene-1,1-diphosphonic acid (1).

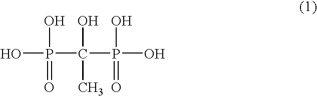

The salts are preferably the sodium, potassium, lithium, ammonium or substituted ammonium salts of the specified phosphonic acids.

The formulations according to the invention can comprise 25 to 90% by weight, preferably 50 to 85% by weight, particularly preferably 60 to 80% by weight and very particularly preferably 70 to 80% by weight, of the oligo- or polyesters. The content of phosphonic acid or phosphonate in the formulation according to the invention can be 0.1 to 40% by weight, preferably 0.15 to 25% by weight, particularly preferably 0.2 to 10% by weight and very particularly preferably 0.25 to 5% by weight.

The aqueous oligo- and polyester preparations according to the invention are generally used in detergents and cleaner formulations in concentrations of less than 5% by weight, preferably less than 3% by weight and particularly preferably 0.5 to 1% by weight (concentration data here based on the polyester fraction in the preparation).

The detergent and cleaner formulations in which the aqueous oligo- and polyester preparations according to the invention can be used are preferably liquid detergents, washing gels and washing pastes and also softeners or liquid laundry conditioners with which textiles can be treated in the rinse cycle.

The specific polyester preparations can easily be incorporated into these liquid formulations using mechanical homogenization devices such as stirrers. The aqueous polyester preparations can, moreover, be used in special laundry treatment compositions, such as, for example, stain dissolvers, stain sprays or in washing boosters.

Liquid formulations comprising the aqueous oligo- and polyester preparations can be packaged in films which either have a protective function during storage or else serve as metering aid. The films may be water-soluble.

The detergents and cleaners or laundry treatment compositions which comprise the aqueous oligo- or polyester preparations according to the invention can, moreover, comprise further customary constituents. These are described below:

### Anionic Surfactants

Suitable anionic surfactants are sulfates, sulfonates, carboxylates, phosphates and mixtures thereof. Suitable cations here are alkali metals, such as, for example, sodium or potassium, or alkaline earth metals, such as, for example, calcium or magnesium, and also ammonium, substituted ammonium compounds, including mono-, di- or triethanolammonium cations, and mixtures thereof.

The following types of anionic surfactants are particularly preferred:

alkyl ester sulfonates, alkyl sulfates, alkyl ether sulfates, alkylbenzenesulfonates, alkanesulfonates and soaps, as described below.

Alkyl ester sulfonates are inter alia linear esters of C₈-C₂₀-carboxylic acids (i.e. fatty acids) which are sulfonated by means of gaseous SO₃, as described in “The Journal of the American Oil Chemists Society” 52 (1975), pp. 323-329.

Suitable starting materials are natural fats, such as, for example, tallow, coconut oil and palm oil, but may also be synthetic in nature.

Preferred alkyl ester sulfonates, specifically for detergent applications, are compounds of the formula

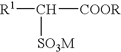

in which R¹ is a C₈-C₂₀-hydrocarbon radical, preferably alkyl, and R is a C₁-C₆-hydrocarbon radical, preferably alkyl. M is a cation which forms a water-soluble salt with the alkyl ester sulfonate. Suitable cations are sodium, potassium, lithium or ammonium cations, such as monoethanolamine, diethanolamine and triethanolamine. Preferably, R¹ is C₁₀-C₁₆-alkyl and R is methyl, ethyl or isopropyl. Particular preference is given to methyl ester sulfonates in which R¹ is C₁₀-C₁₆-alkyl.

Here, alkyl sulfates are water-soluble salts or acids of the formula ROSO₃M, in which R is a C₁₀-C₂₄-hydrocarbon radical, preferably an alkyl or hydroxyalkyl radical with C₁₀-C₂₀-alkyl component, particularly preferably a C₁₂-C₁₈-alkyl or hydroxyalkyl radical.

M is hydrogen or a cation, e.g. an alkali metal cation (e.g. sodium, potassium, lithium) or ammonium or substituted ammonium, e.g. methyl-, dimethyl- and trimethylammonium cations and quaternary ammonium cations such as tetramethylammonium and dimethylpiperidinium cations and quaternary ammonium cations derived from alkylamines, such as ethylamine, diethylamine, triethylamine and mixtures thereof.

Alkyl chains with C₁₂-C₁₆ are preferred for low washing temperatures (e.g. below ca. 50° C.) and alkyl chains with C₁₆-C₁₈ are preferred for higher washing temperatures (e.g. above ca. 50° C.).

Alkyl ether sulfates are water-soluble salts or acids of the formula RO(A)ₘSO₃M, in which R is an unsubstituted C₁₀-C₂₄-alkyl or hydroxyalkyl radical, preferably a C₁₂-C₂₀ alkyl or hydroxyalkyl radical, particularly preferably C₁₂-C₁₈-alkyl or hydroxyalkyl radical.

A is an ethoxy or propoxy unit, m is a number greater than 0, preferably between ca. 0.5 and ca. 6, particularly preferably between ca. 0.5 and ca. 3 and M is a hydrogen atom or a cation, such as, for example, sodium, potassium, lithium, calcium, magnesium, ammonium or a substituted ammonium cation. Specific examples of substituted ammonium cations are methyl-, dimethyl-, trimethylammonium and quaternary ammonium cations, such as tetramethylammonium and dimethylpiperidinium cations and also those which are derived from alkylamines, such as ethylamine, diethylamine, triethylamine or mixtures thereof. Examples which may be mentioned are C₁₂-C₁₈-fatty alcohol ether sulfates, where the content of EO is 1, 2, 2.5, 3 or 4 mol per mole of the fatty alcohol ether sulfate, and in which M is sodium or potassium.

In secondary alkanesulfonates, the alkyl group can either be saturated or unsaturated, branched or linear and optionally substituted by a hydroxyl group. The sulfo group can be at any desired position on the carbon chain, the primary methyl groups having no sulfonate groups at the start of the chain and at the end of the chain.

The preferred secondary alkanesulfonates contain linear alkyl chains having ca. 9 to 25 carbon atoms, preferably ca. 10 to ca. 20 carbon atoms and particularly preferably ca. 13 to 17 carbon atoms. The cation is, for example, sodium, potassium, ammonium, mono-, di- or triethanolammonium, calcium or magnesium, and mixtures thereof. Sodium as cation is preferred.

Further suitable anionic surfactants are alkenyl- or alkylbenzenesulfonates. The alkenyl or alkyl group may be branched or linear and optionally substituted by a hydroxyl group. The preferred alkylbenzenesulfonates contain linear alkyl chains having ca. 9 to 25 carbon atoms, preferably from ca. 10 to ca. 13 carbon atoms, the cation is sodium, potassium, ammonium, mono-, di- or triethanolammonium, calcium or magnesium and mixtures thereof.

For mild surfactant systems, magnesium as cation is preferred, whereas for standard washing applications, sodium is preferred. The same is true for alkenylbenzenesulfonates.

The term anionic surfactants also includes olefinsulfonates which are obtained by sulfonation of C₈-C₂₄—, preferably C₁₄-C₁₆-α-olefins with sulfur trioxide and subsequent neutralization. As a consequence of the preparation process, these olefinsulfonates can comprise relatively small amounts of hydroxyalkanesulfonates and alkanedisulfonates.

Further preferred anionic surfactants are carboxylates, e.g. fatty acid soaps and comparable surfactants. The soaps may be saturated or unsaturated and can contain various substituents, such as hydroxyl groups or α-sulfonate groups. Preference is given to linear saturated or unsaturated hydrocarbon radicals as hydrophobic fraction with ca. 6 to ca. 30, preferably ca. 10 to ca. 18, carbon atoms.

Suitable anionic surfactants are also salts of acylaminocarboxylic acids, the acyl sarcosinates which are formed by reacting fatty acid chlorides with sodium sarcosinate in an alkaline medium; fatty acid-protein condensation products which are obtained by reacting fatty acid chlorides with oligopeptides; salts of alkyl-sulfamidocarboxylic acids; salts of alkyl- and alkylaryl ether carboxylic acids; alkyl- and alkenyl glycerol sulfates such as oleylglycerol sulfates, alkylphenol ether sulfates, alkyl phosphates, alkyl ether phosphates, isethionates, such as acyl isethionates, N-acyl taurides, alkyl succinates, sulfosuccinates, monoesters of sulfosuccinates (particularly saturated and unsaturated C₁₂-C₁₈-monoesters) and diesters of sulfosuccinates (particularly saturated and unsaturated C₁₂-C₁₈-diesters), acyl sarcosinates, sulfates of alkyl polysaccharides such as sulfates of alkyl polyglycosides, branched primary alkyl sulfates and alkyl polyethoxycarboxylates such as those of the formula RO(CH₂CH₂)ₖCH₂COO⁻M⁺, in which R is C₈ to C₂₂-alkyl, k is a number from 0 to 10 and M is a cation.

### Nonionic Surfactants.

Condensation products of aliphatic alcohols with ca. 1 to ca. 25 mol of ethylene oxide.

The alkyl chain of the aliphatic alcohols may be linear or branched, primary or secondary, and generally comprises ca. 8 to ca. 22 carbon atoms. Particular preference is given to the condensation products of C₁₀-C₂₀-alcohols with ca. 2 to ca. 18 mol of ethylene oxide per mole of alcohol. The alkyl chain may be saturated or else unsaturated. The alcohol ethoxylates can have a narrow (“narrow range ethoxylates”) or a broad homolog distribution of the ethylene oxide (“broad range ethoxylates”). Examples of commercially available nonionic surfactants of this type are Tergitol® 5-S-9 (condensation product of a linear secondary C₁₁-C₁₅-alcohol with 9 mol of ethylene oxide), Tergitol® 24-L-NMW (condensation product of a linear primary C₁₂-C₁₄-alcohol with 6 mol of ethylene oxide for a narrow molecular weight distribution). This product class likewise includes the Genapol® grades from Clariant GmbH.

Condensation products of ethylene oxide with a hydrophobic base, formed by condensation of propylene oxide with propylene glycol.

The hydrophobic moiety of these compounds preferably has a molecular weight between ca. 1500 and ca. 1800. The addition of ethylene oxide onto this hydrophobic moiety leads to an improvement in the water solubility. The product is liquid up to a polyoxyethylene content of ca. 50% of the total weight of the condensation product, which corresponds to a condensation with up to ca. 40 mol of ethylene oxide. Commercially available examples of this product class are the Pluronic® grades from BASF and the ®Genapol PF grades from Clariant GmbH.

Condensation products of ethylene oxide with a reaction product of propylene oxide and ethylenediamine.

The hydrophobic unit of these compounds consists of the reaction product of ethylenediamine with excess propylene oxide and generally has a molecular weight of ca. 2500 to 3000. Ethylene oxide is added onto this hydrophobic unit up to a content of ca. 40 to ca. 80% by weight of polyoxyethylene and a molecular weight of ca. 5000 to 11 000. Commercially available examples of this compound class are the ®Tetronic grades from BASF and the ®Genapol PN grades from Clariant GmbH.

### Semipolar Nonionic Surfactants

This category of nonionic compounds includes water-soluble amine oxides, water-soluble phosphine oxides and water-soluble sulfoxides, in each case with an alkyl radical of ca. 10 to ca. 18 carbon atoms. Semipolar nonionic surfactants are also amine oxides of the formula

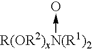

R here is an alkyl, hydroxyalkyl or alkylphenol group with a chain length of ca. 8 to ca. 22 carbon atoms, R² is an alkylene or hydroxyalkylene group with ca. 2 to 3 carbon atoms or mixtures thereof, each radical R¹ is an alkyl or hydroxyalkyl group with ca. 1 to ca. 3 carbon atoms or a polyethylene oxide group with ca. 1 to ca. 3 ethylene oxide units and x is a number from 0 to about 10. The R¹ groups can be joined together via an oxygen or nitrogen atom and thus form a ring. Amine oxides of this type are particularly C₁₀-C₁₈-alkyldimethylamine oxides and C₈-C₁₂-alkoxyethyl dihydroxyethylamine oxides.

### Fatty Acid Amides

Fatty acid amides have the formula

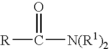

in which R is an alkyl groups with ca. 7 to ca. 21, preferably ca. 9 to ca. 17 carbon atoms and each radical R¹ is hydrogen, C₁-C₄-alkyl, C₁-C₄-hydroxyalkyl or (C₂H₄O)ₓH, where x varies from ca. 1 to ca. 3. Preference is given to C₈-C₂₀-amides, -monoethanolamides, -diethanolamides and -isopropanolamides.

Further suitable nonionic surfactants are alkyl and alkenyl oligoglycosides and fatty acid polyglycol esters or fatty amine polyglycol esters having in each case 8 to 20, preferably 12 to 18, carbon atoms in the fatty alkyl radical, alkoxylated triglycamides, mixed ethers or mixed formyls, alkyl oligoglycosides, alkenyl oligoglycosides, fatty acid N-alkylglucamides, phosphine oxides, dialkyl sulfoxides and protein hydrolyzates.

Polyethylene oxide, polypropylene oxide and polybutylene oxide condensates of alkylphenols.

These compounds include the condensation products of alkylphenols with a C₆- to C₂₀-alkyl group, which may either be linear or branched, with alkylene oxides. Preference is given to compounds with ca. 5 to 25 mol of alkene oxide per mole of alkylphenol. Commercially available surfactants of this type are, for example, Igepal® CO-630, Triton® X-45, X-114, X-100 and X102, and the ®Arkopal-N grades from Clariant GmbH. These surfactants are referred to as alkylphenol alkoxylates, for example alkylphenol ethoxylates.

### Zwitterionic Surfactants

Typical examples of amphoteric or zwitterionic surfactants are alkylbetaines, alkylamidobetaines, aminopropionates, aminoglycinates, or amphoteric imidazolinium compounds of the formula

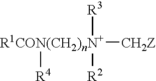

in which R¹ is C₈-C₂₂-alkyl or -alkenyl, R² is hydrogen or CH₂CO₂M, R³ is CH₂CH₂OH or CH₂CH₂OCH₂CH₂CO₂M, R⁴ is hydrogen, CH₂CH₂OH or CH₂CH₂COOM, Z is CO₂M or CH₂CO₂M, n is 2 or 3, preferably 2, M is hydrogen or a cation such as alkali metal, alkaline earth metal, ammonium or alkanolammonium.

Preferred amphoteric surfactants of this formula are monocarboxylates and dicarboxylates. Examples thereof are cocoamphocarboxypropionate, cocoamidocarboxypropionic acid, cocoamphocarboxyglycinate (also referred to as cocoamphodiacetate) and cocoamphoacetate.

Further preferred amphoteric surfactants are alkyldimethylbetaines (® Genagen LAB/Clariant GmbH) and alkyldipolyethoxybetaines with an alkyl radical having ca. 8 to ca. 22 carbon atoms, which may be linear or branched, preferably having 8 to 18 carbon atoms and particularly preferably having ca. 12 to ca. 18 carbon atoms.

### Cationic Surfactants

Suitable cationic surfactants are substituted or unsubstituted straight-chain or branched quaternary ammonium salts of the type R¹N(CH₃)₃⁺X⁻, R¹R²N(CH₃)₂⁺X⁻, R¹R²R³N(CH₃)⁺X⁻ or R¹R²R³R⁴N⁺X⁻. The radicals R¹, R², R³ and R⁴ can preferably, independently of one another, be unsubstituted alkyl with a chain length between 8 and 24 carbon atoms, in particular between 10 and 18 carbon atoms, hydroxyalkyl having ca. 1 to ca. 4 carbon atoms, phenyl, C₂-C₁₈-alkenyl, C₇-C₂₄-aralkyl, (C₂H₄O)ₓH, where x is from ca. 1 to ca. 3, alkyl radicals containing one or more ester groups or cyclic quaternary ammonium salts. X is a suitable anion.

### Builders

Builders can be present in the detergent and cleaner compositions in weight fractions of from about 5% to about 60%. Inorganic builders include, for example, alkali metal, ammonium and alkanolammonium salts of polyphosphates, such as, for example, sodium tripolyphosphate, sodium pyrophosphate and sodium orthophosphate; silicates; carbonates including bicarbonates and sesquicarbonates; sulfates and aluminosilicates. Examples of silicate builders are the alkali metal silicates, in particular those with an SiO₂:Na₂O ratio between 1.6:1 and 3.2:1. Aluminosilicate builders are also zeolites with the formula Na_{z}[(AlO₂)_{z}(SiO₂)_{y}].xH₂O, in which z and y are integers of at least 6, the ratio of z to y is between 1.0 to about 0.5 and x is an integer of from about 15 to about 264. Aluminosilicates may be of crystalline or amorphous structure and be naturally occurring or else prepared synthetically. Preferred ion exchangers based on synthetic crystalline aluminosilicates are available under the name zeolite A, zeolite P(B) and zeolite X.

Important organic builders (cobuilders) are polycarboxylates, e.g. based on acrylic acid and maleic acid (Sokalan CP grades/BASF); builders based on citrate, e.g. citric acid and its soluble salts, in particular Na citrate; phosphonates, such as ethane-1-hydroxy-1,1-diphosphonate and the other phosphonates mentioned at the start.

In detergents and cleaners, these phosphonates have the function of improving the primary detergency toward certain soilings. In detergents containing bleach, they improve the storage stability of the bleach by binding heavy metal ions. However, this object corresponding to the prior art is in no way related to the use according to the invention of the phosphonates in aqueous oligo- and polyester preparations for preventing cloudiness and precipitates.

Further suitable organic builders include polycarboxyl compounds, such as, for example, ether polycarboxylates and oxydisuccinates (as described, for example, in U.S. Pat. No. 3,128,287 and U.S. Pat. No. 3,635,830); “TMS/TDS” builders (see U.S. Pat. No. 4,663,071); ether hydroxypolycarboxylates; copolymers of maleic anhydride with ethylene or vinyl methyl ether; alkali metal salts, ammonium salts and substituted ammonium salts of polyacetic acids, such as, for example, ethylenediaminetetraacetic acid and nitrilotriacetic acid.

The detergents and cleaners and laundry treatment compositions which comprise the aqueous oligo- and polyester formulations can also comprise the customary auxiliaries which boost the cleaning effect, serve to care for the textile to be washed or alter the use properties of the detergent composition. Suitable auxiliaries comprise, for example, enzymes, in particular proteases, lipases, cellulases, amylases, mannanases, glycosidases; enzyme stabilizers; foam boosters; defoamers; tarnish and/or corrosion inhibitors; dispersants; graying inhibitors; dyes; color transfer inhibitors; fillers; optical brighteners; disinfectants; alkalis; hydrotropic compounds; antioxidants; perfumes; solvents; solubility promoters; processing auxiliaries; plasticizers and antistats.

In addition, the detergents in which the aqueous oligo- and polyester formulations are used can also comprise one or more conventional bleaches, bleach activators, bleach catalysts and suitable stabilizers. For liquid detergents, in order to ensure adequate storage stability, two-chamber bottles are used. In general, it must be ensured that the bleaches used are compatible with the cleaner ingredients. In principle, it is possible to use: peroxy acids, either as free peroxy acid, or it is possible to use a combination of a, an inorganic persalt, for example sodium perborate or sodium percarbonate, or b, hydrogen peroxide with an organic peroxy acid precursor (bleach activator). Examples of peroxy acids include peroxydodecanedioic acid (DPDA), the nonylamide of peroxysuccinic acid (NAPSA), the nonylamide of peroxyadipic acid (NAPAA) and decyldiperoxysuccinic acid (DDPSA), nonanoylamidocaproyloxybenzenesulfonic acid and alkanoyloxybenzenesulfonic acids such as nonanoyloxybenzenesulfonic acid (NOBS) and lauroyloxybenzenesulfonic acid (LOBS) and phthalimidoperoxycaproic acid (PAP). For the use of the oligo- and polyester formulations in liquid detergents and liquid laundry treatment compositions in two-chamber bottles, particular preference is given to bleaching systems based on hydrogen peroxide and the bleach activator tetraacetylethylenediamine (TAED).

A further field of application of the oligo- and polyester formulations according to the invention is the treatment (e.g. the finishing) of synthetic fibers, in particular polyester fibers, or fabrics which contain synthetic fibers, in particular polyester fibers, in the textile industry. Furthermore, the oligo- and polyester formulations according to the invention can also be used in cosmetic preparations such as skin cleansing compositions, for example in shower gels, shampoos, soaps and in skincare compositions.

### EXAMPLES

1. A 70% strength aqueous preparation of the polyester TexCare SRN-100 with the addition of 0.5% by weight (active ingredient) of the phosphonic acid Dequest 2010 was prepared. For this, the polyester was melted and the water with the predissolved phosphonic acid was stirred into the melt. The mixture was afterstirred for 15 minutes and then cooled to room temperature with stirring. To investigate the thermal storage stability, the polyester preparation was stored at 40° C. and visually assessed over a period of 6 months.

For comparison, the polyester preparation was prepared without the addition of the phosphonic acid or with the addition of Sokalan CP 12 S and assessed.

TABLE 1
Stability of an aqueous, 70% strength polyester preparation with
the addition of 0.5% by weight of (active ingredient) Dequest 2010.
Comparative example without Dequest 2010 or with Sokalan
CP 12 S.

| 70% strength polyester | visual assessment | | |
|---|---|---|---|
| preparation from | after the | | |
| TexCare SRN-100 | preparation | after 3 months | after 6 months |
| without addition | clear, | sediment | sediment |
| | homogeneous | | |
| with Sokalan CP 12 S | clear, | clear, | cloudiness |
| | homogeneous | homogeneous | |
| with Dequest 2010 | clear, | clear, | clear, |
| | homogeneous | homogeneous | homogeneous |

2. An 80% strength aqueous preparation of the polyester TexCare SRN-100 with the addition of 0.5% by weight (active ingredient) of the phosphonic acid Dequest 2010 was prepared. For this, the polyester was melted and the water with the predissolved phosphonic acid was stirred into the melt. The mixture was after-stirred for 1 hour and then cooled to room temperature with stirring.

To investigate the thermal storage stability, the polyester preparation was stored at 40° C. and visually assessed after 6 months. For comparison, the polyester preparation was prepared without the addition of the phosphonic acid or with the addition of Sokalan CP 12 S and assessed.

TABLE 2
Stability of an aqueous, 80% strength polyester preparation with the
addition of 0.5% by weight (active ingredient) of Dequest 2010.
Comparative example without Dequest 2010 or with Sokalan
CP 12 S.

| 80% strength polyester | visual assessment | | |
|---|---|---|---|
| preparation from | after the | | |
| TexCare SRN-100 | preparation | after 3 months | after 6 months |
| without addition | clear, | sediment | sediment |
| | homogeneous | | |
| with Sokalan CP 12 S | clear, | clear, | cloudiness |
| | homogeneous | homogeneous | |
| with Dequest 2010 | clear, | clear, | clear, |
| | homogeneous | homogeneous | homogeneous |

### List of Tradenames Used:

| | |
|---|---|
| TexCare ® SRN-100(Clariant) | nonionic soil release polyester, |
| | 100% strength |
| Sokalan ® CP 12 S (BASF) | acrylic acid-maleic acid copolymer, |
| | MM = 3000 g/mol, 50% strength. |
| Dequest ® 2010 (Dequest) | hydroxyethylidene-1,1-diphosphonic |
| | acid, 60% strength |

1. A highly concentrated aqueous formulation of oligo- and polyesters, comprising 25 to 90% by weight of an oligo- and polyester and 0.1 to 40% by weight of a phosphonic acid or salts thereof.

2. The formulation as claimed in claim 1, where the oligo- and polyester is a water-soluble or water-dispersible polyester.

3. The formulation as claimed in claim 1, where the oligo- and polyester is a water-soluble or water-dispersible polyester with a molar mass of less than 20,000.

4. The formulation as claimed in claim 1, where the oligo- and polyester is a water-soluble or water-dispersible polyester which is terminally capped by alkyl polyalkylene glycols.

5. The formulation as claimed in claim 1, where the oligo- and polyester is a water-soluble or water-dispersible polyester which is terminally capped by methyl polyethylene glycols, where the number of ethylene glycol units is </=90.

6. The formulation as claimed in claim 1, where the oligo- and polyester is a water-soluble or water-dispersible polyester which has </=60% by mass of esterified terephthalic acid units.

7. The formulation as claimed in claim 1, where the oligo- and polyester is a water-soluble or water-dispersible polyester which contains ethylene glycol or polyethylene glycol units.

8. The formulation as claimed in claim 1, where the oligo- and polyester is a water-soluble or water-dispersible polyester which contains propylene glycol or polypropylene glycol units.

9. The formulation as claimed in claim 1, where the oligo- and polyester is a water-soluble or water-dispersible polyester which comprises both ethylene glycol or polyethylene glycol and also propylene glycol or polypropylene glycol units.

10. The formulation as claimed in claim 1, where the oligo- and polyester is a water-soluble or water-dispersible polyester which is anionically modified by sulfoisophthalic acid and/or glycerol sulfoethyl ether units and/or glycerol sulfopropyl ether units.

11. The formulation as claimed in claim 1, where the oligo- and polyester is a water-soluble or water-dispersible polyester which is terminally capped by hydroxyethanesulfonic acid, hydroxypropanesulfonic acid or reaction products thereof with ethylene oxide or ethylene glycol or oligomers thereof and/or propylene oxide or propylene glycol or oligomers thereof.

12. The formulation as claimed in claim 1, further comprising (poly)phosphonic acid, aminotrimethylenephosphonic acid (ATMP), ethylene diaminetetra-methylenephosphonic acid, hexamethylenediaminetetra-methylenephosphonic acid, diethylenetriaminepentamethylenephosphonic acid, polyaminomethylenephosphonic acid, hexamethylenetriaminepenta-methylenephosphonic acid, (poly)vinylphosphonic acid or 1-hydroxyethylidene-1,1-diphosphonic acid or Li, Na, K, ammonium or substituted ammonium salts thereof.

13. The formulation as claimed in claim 1, comprising 50 to 85% by weight, of the oligo- and/or polyester.

14. The formulation as claimed in claim 1, comprising 0.15 to 25% by weight, of phosphonic acid or salts thereof.

15. A detergent or cleaner comprising a formulation as claimed in claim 1.

16. A cosmetic preparation comprising a formulation as claimed in claim 1.

17. A textile auxiliary for the treatment of synthetic fibers comprising a formulation as claimed in claim 1.

18. The formulation as claimed in claim 1, where the oligo- and polyester is a water-soluble or water-dispersible polyester with a molar mass of less than 10,000.

19. The formulation as claimed in claim 1, where the oligo- and polyester is a water-soluble or water-dispersible polyester with a molar mass of less than 5,000.

20. The formulation as claimed in claim 1, where the oligo- and polyester is a water-soluble or water-dispersible polyester which is terminally capped by methyl polyethylene glycols, where the number of ethylene glycol units is </=50.

21. The formulation as claimed in claim 1, where the oligo- and polyester is a water-soluble or water-dispersible polyester which is terminally capped by methyl polyethylene glycols, where the number of ethylene glycol units is </=20.

22. The formulation as claimed in claim 1, where the oligo- and polyester is a water-soluble or water-dispersible polyester which has </=50% by mass of esterified terephthalic acid units.

23. The formulation as claimed in claim 1, comprising 60 to 80% by weight of the oligo- and/or polyester.

24. The formulation as claimed in claim 1, comprising 70 to 80% by weight of the oligo- and/or polyester.

25. The formulation as claimed in claim 1, comprising 0.2 to 10% by weight of phosphonic acid or salts thereof.

26. The formulation as claimed in claim 1, comprising 0.25 to 5% by weight of phosphonic acid or salts thereof.

27. A textile auxiliary for the treatment of textile fabrics containing synthetic fibers comprising a formulation as claimed in claim 1.

28. A textile auxiliary for the treatment of textile fabrics containing polyester fibers comprising a formulation as claimed in claim 1.
